(19) United States
(12) Patent Application Publication
(10) Pub. No.: US 2004/0259249 A1
(43) Pub. Date: Dec. 23, 2004

(54) Method of making stem cells from differentiated cells

(76) Inventors: Nikolai Strelchenko, DeForest, WI (US); Yury Verlinski, Chicago, IL (US)

Correspondence Address: Monique A. Morneault, Esq., Wallenstein Wagner & Rockey, Ltd., 53rd Floor, 311 South Wacker Drive, Chicago, IL, 60606-6630, US

(21) Appl. No.: 10/844,790

(22) Filed: May 13, 2004

Related U.S. Application Data
(60) Provisional application No. 60/471,163, filed on May 16, 2003.

(51) Int. Cl. C12N 5/08; C12N 15/02

(52) U.S. Cl. 435/449; 435/372

(57) ABSTRACT

Disclosed are methods of creating a stem cell. Specifically described are methods of creating a pluripotent stem cell by reprogramming a differentiated cell. The method of creating the stem cell comprises the steps of obtaining a cytoplast from an existing embryonic stem cell; fusing the cytoplast with a differentiated cell to produce a cybrid; and culturing the cybrid to yield a pluipotent stem cell.

### CROSS-REFERENCE TO RELATED APPLICATIONS

The present application claims priority to U.S. Provisional Patent Application No. 60/471,163 filed May 16, 2003 hereby incorporated herein by reference in its entirety.

### TECHNICAL FIELD

This invention relates generally to creating stem cells. More particularly, the present invention relates to a method of creating a stem cell by fusing the cytoplast of an existing embryonic stem cell with a differentiated cell. The resultant cybrid and its progeny exhibit stem cell specific markers. Specifically, the resultant cybrid and its progeny exhibit pluripotent stem cell specific markers. It is believed that stem cells exhibit the same expression of human leukocyte antigens (HLA) as the differentiated cell.

### BACKGROUND OF THE INVENTION

Stem cells are unspecialized or undifferentiated cells that have the unique ability to give rise to many different cell types such as skin, neuron, or other organ cells. They also possess the ability to self-replicate for indefinite periods of time. The fact that stem cells have the potential of developing into a specific types of cells and can proliferate indefinitely, makes stem cells potentially useful in applications such as developing organs and tissues for transplantation, cell therapies for degenerative diseases, gene therapy, and toxicology testing of new drugs.

Embryonic stem cells originate from one of the earliest stages of development of the embryo, the blastocyst stage. More specifically, embryonic stem cells derive from the inner cell mass of the blastocyst at the stage before it would implant in the uterine wall. Most importantly, embryonic stem cells are pluripotent which means they are capable of self-renewal and differentiating into most any type of cell in the body including the cells of all three germ layers.

In contrast to embryonic stem cells, there also exist adult stem cells. An adult stem cell is an unspecialized or undifferentiated cell that is found in differentiated tissue. The adult stem cell has the ability to differentiate into the cell type from the tissue it originated. Adult stem cells can be found in bone marrow, the blood stream, the cornea and the retina of the eye, dental pulp of the tooth, liver, skin, gastrointestinal tract, and the pancreas. However, unlike embryonic stem cells, there is no evidence that adult stem cells are pluripotent. Thus, there is a need to create pluripotent stem cells.

One source of human embryonic stem cells is derived from embryos created through in vitro fertilization (IVF). IVF technology has made it possible to carry out fertilization and grow embryos in the laboratory. Traditionally, this technology has allowed for many otherwise infertile couples to have children. However, in many cases not all the embryos created are used, and the remaining embryos may frozen and stored. The embryos offer a potential source for human embryonic stem cells. Alternatively, embryonic stem cells may also be created using nuclear transplantation, known as therapeutic cloning.

However, the source of human embryonic stem cells, from surplus embryos from in vitro fertilization, and their use, has raised ethical and policy issues. Techniques such as IVF technology and therapeutic cloning have been developed to address some of these issues; however, concerns still exist with using these techniques.

The present invention is based on the belief that cytoplast of an existing embryonic stem cell can reprogram the nucleus of a differentiated cell into a pluripotent stem cell. It is further believed that the resultant cybrid is immunologically compatible with the donor differentiated cell. The newly created pluripotent stem cell line will differentiate into cells with the same expression of human leukocyte antigens (HLA) as the source of the nucleus, thereby avoiding the issue of potential immunological rejection in patients. Therefore, the present invention is designed to solve the issues addressed above as well as other problems.

### SUMMARY OF THE INVENTION

The present invention provides a method of creating a stem cell by reprogramming a differentiated cell into a stem cell. The method of creating the stem cell comprises the steps of: obtaining a cytoplast from an existing embryonic stem cell; fusing the cytoplast with a differentiated cell to produce a cybrid; and culturing the cybrid to yield a stem cell. The resultant cybrid is a pluripotent stem cell.

### BRIEF DESCRIPTION OF THE DRAWINGS

FIG. 1 shows an existing line of embryonic stem cells growing on a thin layer of growth medium. (objective 10×);

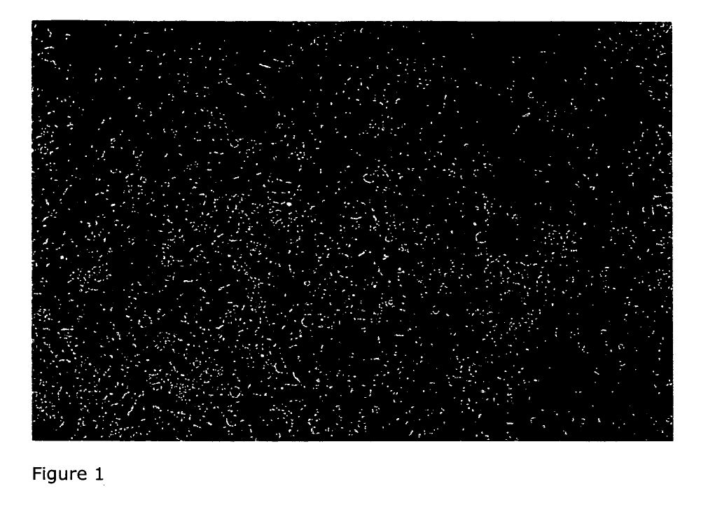

FIG. 2 shows existing embryonic stem cells after enucleation and stained with Hoechst 33342. (objective 20×); and,

FIG. 3 shows stem cell colony after fusion of lymphocytes with embryonic stem cell cytoplast after one to two weeks of culturing. (objective 20×).

### DETAILED DESCRIPTION

While the present invention is susceptible of embodiment in many different forms, this disclosure will describe in detail at least one preferred embodiment, and possible alternative embodiments, of the invention with the understanding that the present disclosure is to be considered merely as an exemplification of the principles of the invention and is not intended to limit the broad aspect of the invention to the specific embodiments illustrated.

The present invention relates to creating a stem cell by fusing cytoplast from an existing embryonic stem cell with a differentiated donor cell to yield a cybrid, also referred to as a “stem cybrid,” that is a pluripotent stem cell. It is believed that the resultant stem cybrid expresses the Major Histocompatibility Complex (MHC) of the differentiated donor cell, specifically, the same expression of human leukocyte antigens (HLA) as the source of the differentiated cell.

An existing embryonic stem cell line is used in the present invention. Differentiated diploid mammalian cells may be obtained by well known methods. Preferably, this invention uses mammalian diploid cells with low amounts of cytoplast relative to karyoplast or nuclei. In one embodiment of this invention diploid mammalian lymphocytes are used, because lymphocytes have relatively small amounts of cytoplast in relation to its karyotype. Typically, lymphocytes have a very thin rim of cytoplast surrounding the karyoplast or nuclei of the cell. It is desirable to use differentiated cells with very low amounts of cytoplast for fusion with existing embryonic stem cell cytoplast. Because it is believed there are certain cytoplasmic factors that alter the phenotypic expression of a host nuclei, it is preferable to have more donor cell cytoplast relative to the differentiated cell cytoplast. Human diploid lymphocytes can be obtained from heparinized blood from humans using known methods, then diluted with Ca,Mg free phosphate buffer solution (PBS).

A suspension of embryonic stem cells is prepared by disaggregating cell colonies in ethylene diamine tetra acetate (EDTA) or EGTA (0.02% in Ca, Mg free PBS or by Hank's solution), and subsequent filtering through 100 μM Nylon cell strainer. Clusters of embryonic stem cells are grown overnight in about an 80% confluent density on a round 18 mm cover slip coated with a growth medium, such as Matrigel. The embryonic stem cells are incubated for about at least 18 hours. After the embryonic cells are disaggregated, the embryonic stem cell is synchronized, such that they are in the same stage of the cell cycle, before cell fusion. The cells may be synchronized in order to increase the efficiency of cell fusion.

A suspension of differentiated cells is prepared by obtaining differentiated mammalian diploid cells using well known methods. In one embodiment of this invention, human diploid cells are prepared using heparinized blood. More preferably, human lymphocytes are prepared using a density gradient. Human heparinized blood is diluted with Ca,Mg free PBS. Preferably, the amount of human heparinized blood to PBS solution is in a 1:2 ratio. After adding the PBS, a reagent, such as Histopaque 1083, is added to the heparinized blood and PBS mixture. Histopaque 1083 is preferably added so there is two times (2×) the amount of Histopaque as there is volume of heparinzed blood. Histopaque 1083 is a reagent that is adjusted to the density of 1.083 g/mL and facilitates the recovery of viable mononuclear cells. The density gradient employed to suspend the lymphocytes is a one-step ladder gradient. After suspending the heperanized blood in PBS and Histopque, the suspension is centrifuged for about 30 minutes at 600 G at room temperature. After centrifugation, the heparinized blood is separated by density, with the red blood cells comprising the pellet at the bottom of the tube, and the lymphocytes the layer above the gradient in the tube. The layer of lymphocytes is removed and treated so as to remove any traces of serum in order to prepare the lymphocytes for cell fusion.

To increase the efficiency of fusion, it is preferred that the existing embryonic stem cells are synchronized before they are enucleated. It is preferred that about 50% of the cells in a population are arrested in one stage of the cycle, more preferably 70% of cells in a population of cells are arrested in one stage of the cell cycle, and most preferably about 90% of cells in a population of cells are arrested in one stage of the cell cycle prior to fusion. The existing embryonic stem cells can be synchronized using known methods. Cell cycle stages are distinguishable based on relative cell size, or by a variety of known cell markers. In the present invention, it is preferred that the population of existing embryonic stem cells are separated based on their relative size. It is further preferred that the population of existing embryonic stem cells are arrested at the G0/G1 stages before enucleation. When cells are in the G0/G1 phase they are not actively proliferating by means of mitotic cell division. Since lymphocytes normally exist in the G0/G0 phase, they did not need to be synchronized before for fusion.

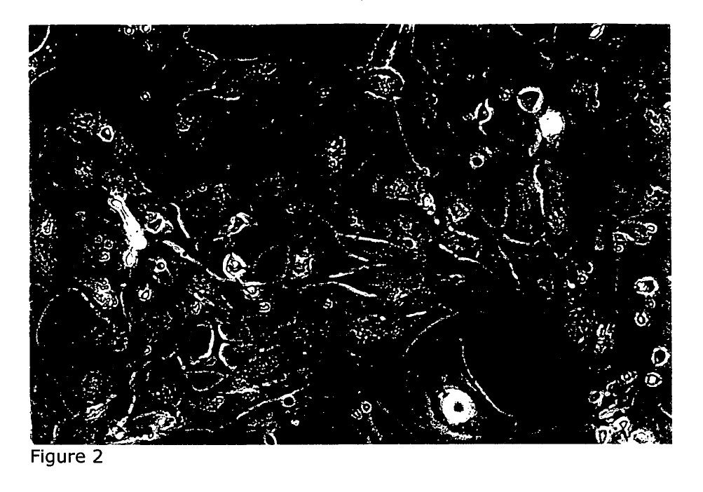

After the existing embryonic stem cells are synchronized in the G0/G1 phase, they are enucleated in order to isolate the cytoplast from the cell. Enucleation is a known technique wherein the nuclei are separated from the cytoplast of a cell. Enucleation of differentiated cells can be achieved by high speed centrifugation in a ladder gradient of ficoll in the presence of Cytochalasin B or Cytochalasin D. Other reagents, including but not limited to, biological or chemical products that disrupt actin filaments in the cytoskeleton may also be used for enucleation. See Wigler & Weinstein (1975) Biochem. Biophys. Res. Commun. 63(3):669-674. Enucleation of the existing embryonic stem cells requires a different technique compared to enucleation of differentiated and adhered cells because embryonic stem cell cytoplast is relatively small compared to its nucleus and cannot be separated in the same manner as differentiated cells such as fibroblasts. Enucleation of embryonic stem cells involves a procedure that uses a high density supported solution that allows the cytoplast to stay anchored to the glass during centrifugation, while the nuclei migrate toward the bottom of the tube. In one embodiment of the invention, the existing embryonic stem cells are enucleated in a one step ladder gradient of ficoll. The density gradient is created by mixing 3.3 mL of 25% ficoll F400 in standard HTF-HEPES solution with 4.7 mL HTF-HEPES and 1.3 mL Serum Replacement. The cell enucleating solution includes 3.4 μg/mL Cystohalisin D and 0.5 μg/mL of nocodasol. The cover glass with the existing embryonic stem cells are placed with the face to the bottom into round centrifuge tubes with no air bubbles under the slip. They are centrifuged in a HB-4 Bucket Rotor at 20,000 G that is pre-warmed to 35° C. and the temperature is maintained at 36° C. during centrifugation for about 1 hour. After centrifugation is complete, the cover slips with the cytoplasts of the existing embryonic stem cells are washed in HTF-HEPES or Hank's solution with 10% fetal bovine serum and transferred into 35 mm Petri dishes with growth media for recovery for 2 to 3 hours. The efficiency of enucleation is then evaluated under an UV-microscope with Hoechst 33342 (3 mkg/mL) as shown in FIG. 2. The parts that are stained are the nuclei, whereas the unstained parts are the cytoplast.

After obtaining the enucleated existing embryonic stem cell cytoplasts and the differentiated cells, such as lymphocytes, fusion of the cell cytoplasts and differentiated cells can be accomplished. In one aspect of this invention, lipid membranes can be fused by electrical or chemical means that are well known to persons of ordinary skill in the art. For example, fusion is accomplished chemically using the chemical fusion reagent, polyethlenglycol (PEG). See Pontecorvo “Polyethylene Glycol (PEG) in the Production of Mammalian Somatic Cell Hybrids” Cytogenet Cell Genet. (1976) 16(1-5): 399-400. It is preferred that the molecular weight range of PEG be of at least about 1500. However, PEG molecules of a wide range of molecular weight can be used for cell fusion.

Fusion of the cytoplast with differentiated cells is accomplished as follows: after the mammalian lymphocytes are isolated from 6 mL of blood, they are washed with Hank's solution and carefully layered on top of the attached existing embryonic stem cell cytoplasts. The suspension is centrifuged at 1500 rpm for 10 minutes to provide a tight contact between the enucleated existing embryonic stem cell cytoplasts and the mammalian lymphocytes. Preferably, a ratio of 1 cytoplast for every 10 lymphocytes is to be used. A fusion agent comprising 40-50% PEG and 5% dimethyl sulfoxide (DMSO) was added to the cover glass and were allowed to incubate at room temperature for 60 to 90 seconds. The fusion agent, PEG, is then diluted by adding additional serum-free medium. The serum-free medium is then replaced with growth medium and the cover glass is transferred into Petri dishes with Matrigel and allowed to proliferate by incubating for about 18 hours at +37° C.

For further proliferation, the stem cybrids are cultured using standard procedures. For example, the covered glass with the adhered stem cybrids are transferred into Petri dishes with a feeder layer of embryonic fibroblasts and allowed to incubate for further proliferation.

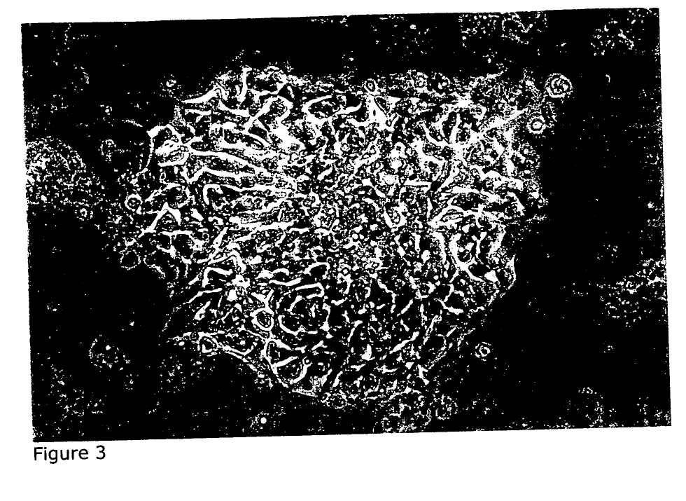

At the end of the second week, primary colonies appeared. The fused stem cybrid, formed from existing embryonic stem cell cytoplasts and mammalian lymphocytes, proliferated further as seen in FIG. 3 and described in Table 1. The existing embryonic stem cell cytoplasts and mammalian lymphocytes that did not fuse, did not proliferate.

Further screening for specific markers relating to pluripotent stem cells were done using hybridization methods, such as fluorescence in situ hybridization (FISH). The results of the screening are shown in Table 2. Table 2 indicates that the resulting stem cybrid contain pluripotent stem cell specific markers.

### EXAMPLES

The examples below are non-limiting and are merely representative of various aspects and features of the presentation.

### Example 1

### Reprogramming of Male Lymphocyte into Pluripotent Stem Cell

Using the above techniques, female cytoplasts of existing cell line 96 from the collection of Reproductive Genetics Institute were used. The existing cell line contains a euploid karyotype 46, XX. Preliminary tested lymphocytes have a diploid karyotype 46, XY. Enucleation of existing embryonic stem cell line 96 was performed as written in the protocol by centrifugation of plated 15×10⁴ existing embryonic stem cells on 18 mm cover glass in high density solution at 20,000 g for 1 hr. After 2 hrs of recovery in cell culture medium existing embryonic stem cell cytoplasts were washed twice in PBS (serum free) and 1.5×10⁶ of preliminary isolated lymphocytes was sedimented on top of the cytoplasts. Cell-cytoplast fusion was performed by 40% PEG 1500 with 5% DMSO.

TABLE 1

| | | | | Reprogrammed |
|---|---|---|---|---|
| Clones | ES-96 2n | Leucocytes 2n | Karyotype | stem cybrid |
| C1 | 46,XX | 46,XY | 46,XX | |
| C2 | 46,XX | 46,XY | 46,XX | |
| C3 | 46,XX | 46,XY | 46,XX | |
| C5 | 46,XX | 46,XY | 46,XX | |
| D2 | 46,XX | 46,XY | 46,XX | |
| D3 | 46,XX | 46,XY | 46,XX | |
| D4 | 46,XX | 46,XY | 46,XX | |
| D5 | 46,XX | 46,XY | 46,XX | |
| D8 | 46,XX | 46,XY | 46,XY | cybrid |
| E1 | 46,XX | 46,XY | 46,XX | |
| E2 | 46,XX | 46,XY | 46,XX | |
| E3 | 46,XX | 46,XY | 46,XX | |
| E4 | 46,XX | 46,XY | 46,XX | |
| E6 | 46,XX | 46,XY | 46,XX | |
| E8 | 46,XX | 46,XY | 46,XY | cybrid |
| F1 | 46,XX | 46,XY | 46,XX | |
| F2 | 46,XX | 46,XY | 46,XX | |
| F3 | 46,XX | 46,XY | 46,XX | |
| F4 | 46,XX | 46,XY | 46,XX | |
| F5 | 46,XX | 46,XY | 46,XY | cybrid |
| F6 | 46,XX | 46,XY | 46,XY | |
| F7 | 46,XX | 46,XY | 46,XX | |
| F8 | 46,XX | 46,XY | 46,XX | |

Isolated clones were screened for pluripotent stem cell specific markers (shown below) using fluorescence in situ hybridization and can be successfully frozen and thawed out.

TABLE 2

| | Alkaline | | | TRA-2- | | TRA- | TRA- |
|---|---|---|---|---|---|---|---|
| Clones | Phosphates | SSEA3 | SSEA4 | 29 | Oct-4 | 1-60 | 1-80 |
| D8 | + | + | + | + | + | + | + |
| E8 | + | + | + | + | + | + | + |
| F5 | + | + | + | + | + | + | + |

Reprogramming and immortalization of human lymphocytes have been performed by fusion with cytoplasts of mouse L-cells enucleated with cytochalasine B by 50% PEG 4000 and further cultured. As result of fusing human lymphocytes with cytoplasts, a reprogrammed lymphoid cell line has been obtained. See, Abken et al., Immortalization of Human Lymphocytes by Fusion with Cytoplasts of Transformed Mouse L Cells, The Journal of Cell Biology, September 1986 (103):795-805.

We claim:

1. A method of creating stem cell comprising the steps of:
a. obtaining a cytoplast from an existing embryonic stem cell;
b. fusing the cytoplast with a differentiated cell, to produce a cybrid;
c. culturing the cybrid to yield a stem cell.

2. The method of claim 1, wherein the existing embryonic stem cell is derived from a mammalian species.

3. The method of claim 1, further comprising the step of arresting the existing embryonic stem cell in G0/G1 phase.

4. The method of claim 1, wherein the step of obtaining the cytoplast comprises the step of enucleating the existing embryonic stem cell.

5. The method of claim 4, wherein the step of obtaining the cytoplast further comprises the step of isolating the cytoplast of the existing embryonic stem cell.

6. The method of claim 1, further comprising the step of enucleating the existing embryonic stem cell to obtain a cytoplast.

7. The method of claim 1, further comprising the step of deriving the differentiated cell from a mammalian species.

8. The method of claim 7, wherein the differentiated cell comprises a human lymphocyte.

9. The method of claim 8, further comprising the step of arresting the human lymphocyte in G0/G1 phase before fusing the cytoplast.

10. The method of claim 9, wherein the step of fusing is accomplished by chemical fusion.

11. The method of claim 1, further comprising the step of transferring the cybrid onto a feeder layer before the step of culturing the cybrid to yield the stem cell.

12. The method of claim 11, wherein the feeder layer comprises a fibroblast feeder layer.

13. The method of claim 1, where in the cybrid is a stem cybrid.

14. The method of claim 1, wherein the stem cell is pluripotent.

15. The method of claim 1, wherein the cybrid expresses human leukocyte antigens (HLA) of the differentiated cell.

16. A method of creating an embryonic stem cell comprising the steps of:
a. obtaining a cytoplast from an existing embryonic stem cell;
b. fusing the cytoplast with a lymphocyte cell, to produce a cybrid;
c. culturing the cybrid to yield a stem cell.

17. The method of claim 16, wherein the existing embryonic stem cell is derived from a mammalian species.

18. The method of claim 16, further comprising the step of arresting the existing embryonic stem cell in G0/G1 phase.

19. The method of claim 16, wherein the step of obtaining the cytoplast comprises the step of enucleating the existing embryonic stem cell.

20. The method of claim 19, wherein the step of obtaining the cytoplast further comprises the step of isolating the cytoplast of the existing embryonic stem cell.

21. The method of claim 16, further comprising the step of enucleating the existing embryonic stem cell to obtain a cytoplast.

22. The method of claim 16, further comprising the step of deriving the lymphocyte cell from a mammalian species.

23. The method of claim 22, further comprising the step of arresting the lymphocyte in G0/G1 phase before fusing the cytoplast.

24. The method of claim 23, wherein the step of fusing is accomplished by chemical fusion.

25. The method of claim 16, further comprising the step of transferring the cybrid onto a feeder layer before the step of culturing the cybrid to yield the stem cell.

26. The method of claim 25, wherein the feeder layer comprises a fibroblast feeder layer.

27. The method of claim 16, wherein the cybrid is a stem cybrid.

28. The method of claim 16, wherein the stem cell is pluripotent.

29. The method of claim 16, wherein the cybrid expresses human leukocyte antigens (HLA) of the differentiated cell.
